(19) United States
(12) Patent Application Publication
(10) Pub. No.: US 2010/0028369 A1
(43) Pub. Date: Feb. 4, 2010

(54) Hazardous substance removing material and method for removing hazardous substance

(75) Inventor: Hiroshi Iwanaga, Kanagawa (JP)

Correspondence Address: AKERMAN SENTERFITT, 8100 BOONE BOULEVARD, SUITE 700, VIENNA, VA, 22182-2683, US

(73) Assignee: FUJIFILM Corporation, Tokyo (JP)

(21) Appl. No.: 12/458,935

(22) Filed: Jul. 28, 2009

(30) Foreign Application Priority Data
Jul. 29, 2008 (JP) 2008-194536
Sep. 26, 2008 (JP) 2008-247287

(51) Int. Cl. A01N 25/10; A01P 1/00

(52) U.S. Cl. 424/178.1

(57) ABSTRACT

It is an object of the present invention to provide a hazardous substance removing material, which efficiently captures hazardous substances derived from microorganisms such as bacteria or viruses and rapidly inactivates them, so as to minimize the their influences on human bodies, and which is able to allow an antibody to be supported on a carrier by a simple method, and which has an improved antibody use efficiency. The present invention provides a hazardous substance removing material consisting of a carrier on which an antibody and a polymer material having an affinity for Fc region of the antibody are supported.

### TECHNICAL FIELD

The present invention relates to a hazardous substance removing material capable of selectively inactivating bacteria or viruses and a method for removing a hazardous substance using the same.

### BACKGROUND ART

In recent years, infectious diseases caused by bacteria, molds, viruses and the like have been recognized as social problems. For instance, there is a concern of mass infection in general public places such as hospitals and public facilities. Particularly in the case of hospital infection, the misuse of antibiotics and the like causes the generation of MRSA (Methicillin-resistant Staphylococcus aureus), for example.

In view of the above, recent buildings are provided with a duct in each room in such a manner that air is circulated through the duct, using an air-conditioner, so as to control the room temperature or other conditions of the whole building. Thus, bacteria, molds, viruses and the like floating in a facility are often diffused into the entire facility through such an air conditioner. Therefore, it is considered that blocking of a route for air-mediated infection is particularly effective. Specifically, a finely woven filter is provided to an air distribution part of an air conditioner, air purifier or the like, such that bacteria, mold, viruses or media therefore such as floating fine objects (e.g., dusts) in air are allowed to be adsorbed to the filter. Alternatively, titanium oxide or a strongly acidic sterilizing zone is provided to the same such that bacteria, molds and viruses passing therethrough are inactivated and removed.

However, upon the removal by adsorption, if a hazardous substance is a bacterium, virus or the like, bacteria having captured with a filter might be detached therefrom so as to be reactivated and affect human bodies. In addition, in the case of a method for inactivating a hazardous substance by allowing the hazardous substance to pass through titanium oxide or a strongly acidic sterilizing zone so as to inactivate the hazardous substance, inactivation is time-consuming to a certain extent and the effects obtained thereby are not always sufficient, which has been problematic.

Japanese Patent No. 3642340 describes a method for removing a hazardous substance in a gas phase atmosphere, using a hazardous substance removing substance in which an antibody is supported on a carrier; which is characterized in that it comprises controlling humidity in the atmosphere around the aforementioned antibody so that the antibody exhibits activity at the humidity. Japanese Patent No. 3642340 also describes that the Fc portion of the antibody is allowed to bind to the carrier, so that Fab that captures a hazardous substance becomes outward against the carrier and the contact probability of Fab with the hazardous substance increases, and thereby the hazardous substance can be efficiently captured. However, even in the method of Japanese Patent No. 3642340, antibody use efficiency is low. Thus, in order to more efficiently use the Fab portion, it has been necessary to carry out a special step for the antibody or the carrier. Hence, this method has been problematic in terms of low productivity.

On the other hand, Japanese Patent Publication (Kohyo) 2004-536290 describes a method for producing an antibody array wherein antibody-binding protein which can specifically recognize Fc region of the antibody is immobilized via covalent binding on a surface of a plane plate base, several specific monoclonal antibodies are bound to the antibody-binding protein at the Fc region so that a pattern is formed, and the immobilized complex of antibody-binding protein and antibody are bound via crosslinking of covalent binding. However, Japanese Patent Publication (Kohyo) 2004-536290 does not relate to a hazardous substance removing material where an antibody is supported.

### DISCLOSURE OF THE INVENTION

It is an object of the present invention to solve the problems of the conventional hazardous substance removing materials. Namely, it is an object of the present invention to provide a hazardous substance removing material, which efficiently captures hazardous substances derived from microorganisms such as bacteria or viruses and rapidly inactivates them, so as to minimize the their influences on human bodies, and which is able to allow an antibody to be supported on a carrier by a simple method, and which has an improved antibody use efficiency. Moreover, it is another object of the present invention to provide a method for efficiently removing a hazardous substance using the aforementioned hazardous substance removing material.

As a result of intensive studies directed towards achieving the aforementioned objects, the present inventor has found that a hazardous substance removing material, which efficiently captures hazardous substances and rapidly inactivates them, so as to minimize their influences on human bodies, and which is able to allow an antibody to be supported on a carrier by a simple method, and which has an improved antibody use efficiency, can be obtained by allowing an antibody and a polymer material having an affinity for Fc region of the antibody to be supported on a carrier, thereby completing the present invention.

The present invention provides a hazardous substance removing material consisting of a carrier on which an antibody and a polymer material having an affinity for Fc region of the antibody are supported.

Preferably, the polymer material is at least one type selected from the group consisting of protein A, protein Q and lectin.

Preferably, the polymer material is lectin.

Preferably, the polymer material is mannose-binding-type lectin.

Preferably, the surface of the carrier is coated with the polymer material.

Preferably, the hazardous substance removing material of the present invention has a hydrophilic polymer as well as the polymer material on the carrier.

Preferably, the hydrophilic polymer has a hydroxyl group, an amino group, an amide group, a carboxylic acid group, or a quaternary amino group.

Preferably, the hazardous substance removing material of the present invention is obtained by forming a layer comprising a polymer material having an affinity for Fc region of an antibody on a carrier and then supporting the antibody on the carrier.

Preferably, the antibody is derived from ostriches.

The present invention further provides a method for removing hazardous substance, which comprises removing a hazardous substance from a gas phase or a liquid phase using the aforementioned hazardous substance removing material of the present invention.

According to the present invention, by allowing an antibody and a polymer material having an affinity for Fc region of the antibody to be supported on a carrier, the effective amount of the supported antibody can be increased, and thus, hazardous substances can be reliably deactivated with a small amount of antibody. Furthermore, according to the present invention, it has become possible to provide a hazardous substance removing material having an improved preservative quality as well as an improved rate of capturing airborne bacteria According to the method of the present invention, an air purifier or a liquid purifier capable of efficiently removing hazardous substances in a gas phase or a liquid phase can be produced, which is thus very useful in the industry.

### PREFERRED EMBODIMENT OF THE INVENTION

Hereinafter, the present invention will be described more in detail.

The hazardous substance removing material of the present invention is characterized in that it consists of a carrier on which an antibody and a polymer material having an affinity for Fc region of the antibody are supported.

### (1) Carrier

A main material which forms a carrier used in the present invention is preferably a fiber comprising, as a main component, at least one selected from the group consisting of cellulose ester, vinylon, acrylic, and polyurethane. In addition, as a main material which forms a carrier, a fiber comprising, as a main component, polyamide is also preferable. According to the present invention, the term “main component” means a component that accounts for 25% or more in terms of mass fraction with respect to the total mass of fibers.

According to the present invention, the term “cellulose ester” refers to a cellulose derivative obtained by esterifying a hydroxyl group of cellulose with an organic acid. Examples of an organic acid used for esterification include fatty carboxylic acids such as acetic acid, propionic acid, and butyric acid and aromatic carboxylic acids such as benzoic acid and salicylic acid. They may be used alone or in combination. The rate of substitution of a hydroxyl group of cellulose with an ester group is not particularly limited; however, it is preferably 60% or more.

According to the present invention, a cellulose acylate fiber is preferable among the group of main materials which form a carrier. The term “cellulose acrylate” used herein refers to cellulose ester in which some or all of hydrogen atoms of a hydroxyl group of cellulose are substituted with an acyl group. Examples of an acyl group include an acetyl group, a propionyl group, and a butylyl group. In terms of structure, a single group among the above examples may be substituted, or two or more acyl groups may be subjected to mixed substitution. The total sum of degrees of acyl group substitution is preferably 2.0 to 3.0, more preferably 2.1 to 2.8, and particularly preferably 2.2 to 2.7. Among them, cellulose acetate, cellulose acetate propionate, or cellulose acetate butylate capable of achieving such degree of substitution is preferable, and cellulose acetate is most preferable. In general, it has been known that a solvent for cellulose acylate varies depending on the degree of esterification. It is also possible to produce a carrier with cellulose acylate having a high esterification rate in advance and then subject the carrier to alkali hydrolysis treatment or the like for hydrophilicization of the surface thereof.

It is possible to form a sufficiently practical hazardous substance removing material consisting of a cellulose acylate fiber. However, in order to further improve strength, dimensional stability, and the like, a carrier may be formed with a mixed fiber (e.g., polyester-based fiber/polyolefin-based fiber/polyamide-based fiber/acrylic-based fiber). When a mixed fiber is used, the mass fraction of a cellulose acylate fiber is preferably 50% or more and more preferably 70% or more.

According to the present invention, a polyamide fiber is preferable among the group of main materials which constitutes a carrier.

According to the present invention, the term “polyamide” refers to a fiber comprising a linear polymer having a chemical structure unit comprising an amide bond.

Among polyamides, a linear aliphatic polyamide, which is a combination of an aliphatic diamine such as ethylenediamine, 1-methylethylenediamine, 1,3-propylenediamine, or hexamethylenediamine and an aliphatic dicarboxylic acid such as malonic acid, succinic acid, or adipic acid, is preferable. Nylon 66 is particularly preferable.

In addition to the above diamine and dicarboxylic acid, aliphatic polyamide comprising a single component or copolymer components selected from among the following examples can be used: lactams such as ε-caprolactam and laurolactam; aminocarboxylic-acids such as aminocaproic acid and aminoundecanoicacid; and para-aminomethyl benzoic acid. Nylon 6 produced using ε-caprolactam alone is particularly preferable.

In addition to the above, the following may be used: an aliphatic polyamide in which cycloaliphatic diamine such as cyclohexanediamine, 1,3-bis(aminomethyl)cyclohexane, or 1,4-bis(aminomethyl)cyclohexane is partially or entirely used as a material aliphatic diamine; and/or an aliphatic polyamide in which cycloaliphatic dicarboxylic acid such as 1,4-cyclohexane dicarboxylic acid, hexahydroterephthalic acid, or hexahydroisophthalic acid is partially or entirely used as dicarboxylic acid.

Further, examples of the above polyamide further include a polyamide with decreased water absorbability and an improved elastic modulus in which aromatic diamine such as aliphatic paraxylylene diamine (PXDA) or metaxylylene diamine (MXDA) and aromatic dicarboxylic acid such as terephthalic acid are partially used as starting materials. Moreover, a polymer having a side chain comprising an amide bond such as polyacrylic acid amide, poly(N-methylacrylic acid amide), or poly(N,N-dimethylacrylic acid amide) may be used.

Among polyamides, nylon 66 or nylon 6 is most preferable. This is because the following properties of such polyamide are preferable to be used as the carrier of the present invention: appropriate hygroscopic properties derived from amide bonds; ease of inducing fiber axis orientation of a molecular chain comprising a long-chain fatty acid having an appropriate length that results in relatively high extensibility; a dynamic and kinetic tendency to not be melted due to high melting temperature and thermal capacity (resistance to melting); flexibility of a molecular chain comprising a long-chain fatty acid; and a tendency to not cause fibrillation or kink band formation (such tendency being imparted as a result of formation of a hydrogen bond between amide bonds), that is to say, repetitive bending and stretching properties.

Preferably, a polyamide in which an amide bond in a chemical structure unit exists on a side chain but not on a main chain can be used. Examples thereof include polyacrylamides such as poly(N-isopropylacrylamide), poly(N,N-dimethylacrylamide), and poly(N-hexylacrylamide). In general, a polymer having a side chain comprising an amide bond has high hydrophilicity and thus tends to be swollen/deformed. Thus, it is preferable that a physically crosslinked polymer be formed with the use of a gelatinization phenomenon or a polymer be hydrophobized by a method comprising introducing an alkyl group, for example.

Likewise, in order to improve strength or dimensional stability, a carrier may be reinforced with other appropriate structural materials such as metals, high-molecular materials, and ceramics. It is desirable that such reinforcing materials be used for a part which is not positioned on the substantially outermost surface of a face to which a hazardous substance removing material is applied (such material being used for, for example, the face located opposite to such face or a core material).

According to the present invention, the term “vinylon” refers to a fiber comprising a linear polymer containing vinyl alcohol units (65% by mass or more) and having a moisture regain of less than 7% obtained at least 1 week after placement of such fiber in an environment at a temperature of 20° C. and at a humidity of 65%. Such fiber may be obtained by formalizing a hydroxyl group of vinyl alcohol. Also, it may be a polymer obtained by subjecting a hydroxyl group to boric acid crosslinking or a non-formalized fiber subjected to a waterproof treatment by a known method such as an alkaline spinning method or a cooled gel spinning method. The above fiber may contain, as non-vinyl-alcohol-unit component, an ethylene chain or a vinyl acetate chain. However, it is preferably a fiber formed with a vinyl alcohol carrier. Further, it is most preferably a non-formalized fiber obtained by cooled gel spinning. This is because a non-formalized fiber has uniform properties and high degree of orientation/crystallization and thus excellent mechanical properties and reliability can be obtained.

In general, vinylon is superior to other fibers in terms of high strength, high elastic modulus, appropriate hydrophilicity, weather resistance, chemical resistance, adhesiveness, and the like. Thus, the preferable properties thereof can be used for the carrier of the present invention.

According to the present invention, the term “acrylic” refers to a fiber comprising recurring units of an acrylonitrile group (mass percentage: 40% or more). Examples thereof include a homopolymer of acrylonitrile; a copolymer of acrylnitrile and a nonionic monomer such as acrylic ester, methacrylic ester, or vinyl acetate; a copolymer of acrylonitrile and an anionic monomer such as vinylbenzenesulfonate or allylsulfonate; and a copolymer of acrylonitrile and a cationic monomer such as vinylpyridine or methylvinylpyridine. A promix fiber which is formed from acrylonitrile and milk casein is included in this category.

In general, an acrylic fiber is produced by an organic solvent wet spinning method. In this method, when a spinning stock solution is formed into a coagulated thread in a coagulating bath, water serving as a coagulant is mixed with the spinning stock solution that is spinning-twisted from a nozzle and a spinning solvent is externally diffused from the spinning-twisted stock solution. At such time, water and an organic solvent (e.g., DMF or DMAc) are mutually diffused such that a polymer deposits, resulting in the formation of a line of coagulated thread having a structure in which many cavities are connected to each other in a net form. In addition, such thread is characterized by deformation of a fiber section caused by volume contraction as a result of diffusion of a solvent into a coagulating bath during coagulation and by formation of concave-convex portions as a result of macrofibril structure formation on the surface thereof. Such fine structure is preferable as a structure of a carrier used in the present invention in terms of an increase in specific surface area or the ease of antibody loading.

An acrylic fiber used in the present invention varies depending on the composition of a starting material polymer, a spinning method, post-treatment conditions during production, and the like. However, in general, a bulky fiber having appropriate hydrophilicity and high weather resistance can be obtained, which is advantageous.

The term “polyurethane” used in the present invention refers to a fiber comprising a linear synthetic polymer in which bonds between monomers or basic substrate polymer units are mainly urethane bonds. Preferably such fiber contains a polyurethane segment at a mass percentage of 85% or more. Preferably, such polyurethane is a block copolymer of segmented polyurethane comprising a soft segment that is soft and have a molecular weight of several thousands and a low melting point and a hard segment that is rigid and have high cohesion and a high melting point. For a soft segment, polyether such as polypropylene glycol or polytetramethylene glycol can be used. For a hard segment, a urethane group formed with 4,4′-diphenylmethane diisocyanate, m-xylene diisocyanate, or the like can be used. Polyurethane is generally characterized by a high elasticity. Also, it is further characterized by good extensibility, high restoring force upon expansion and contraction, antidegradation properties better than those of rubber materials, formation into thin fibers, and the like, although the characteristics thereof vary depending upon differences in terms of a primary structure of a high-molecular chain such as the distribution and chemical structure of each segment and upon differences in terms of a secondary structure derived from different spinning conditions. Thus, when polyurethane is used as a carrier of the present invention, such characteristics can be utilized.

Regarding mechanical and physical properties and dimensional stability of a fiber constituting a carrier, the tensile elastic modulus in a dried state is preferably 25% or more. The term “tensile elastic modulus in a dried state” used herein refers to the degree of elongation at break of a fiber in a tensile test at 20° C., provided that such fiber has been dried for a sufficiently long period of time. In general, a fiber having a tensile elastic modulus in a dried state of 10% or more is preferable for processing such as fabric formation. In order to prevent breakage upon filter processing or practical use (such breakage leading to reduction in filtration efficiency), the tensile elastic modulus is preferably 25% or more, more preferably 30% or more, and most preferably 35% or more.

The official moisture regain of the fiber constituting the carrier is preferably not less than 1.0% to less than 7%, more preferably not less than 3.0% to less than 6.5%, most preferably not less than 5.0% to less than 6.5%. Within the above range of official moisture regain, the expression of the activity of a supported antibody and the mechanical strength, rigidity, dimensional change stability in a use environment (particularly humidity) of a carrier can be achieved. Further, a filter obtained therewith can exhibit high performance and reliability.

In addition, the term “moisture regain” used herein refers to an official moisture regain. The term “official moisture regain” refers to a moisture regain of a fiber that has been left in an environment at 20° C. and at a relative humidity of 65% for long period of time. Moreover, when a fiber is a mixed fiber further containing a different fiber, the term refers to the official moisture regain of the total mixed fibers.

Preferably, the surface of a fiber constituting a carrier has fine concave-convex portions several tens nanometers to several micrometers in size. The shape of a concave-convex portion may be a three-dimensionally shaped groove or ridge which is formed in the direction parallel to the fiber direction or in the direction vertical to the same, that is to say, in a concentric direction with respect to the fiber axis. Such three-dimensionally shaped groove or ridge may exist at an arbitrary proportion or density, provided that an arbitrary angle is formed between such groove or ridge and a line extending in the direction parallel thereto, in the direction vertical thereto, or in the direction between such parallel direction and such vertical direction. A sample obtained by a known method for cellulose acetate fiber spinning is known to have a fiber section having a variable chrysanthemum-like shape as a result of skin layer formation on the surface thereof and depression of a skin layer due to solvent drying. In a preferred embodiment, such concave-convex portions are used for the present invention.

The above fine concave-convex portions several tens nanometers to several micrometers in size may have holes and/or projections. Preferably, such holes or projections have an average diameter of 50 nm to 1 μm. Such holes and projections can be formed by, for example, cavitation of a solution or they can be formed in a spinning step of a method using a solution in which a fine dispersoid is dispersed (e.g., a mixture containing a slurry in which barium sulfate particles are dispersed) or in a subsequent step by a method involving hydrolysis of an acyl group, surface oxidation treatment, or the like (e.g., the exposure of a cellulose portion on the fiber surface with the use of an alkaline water solution followed by generation of microcraters by an enzyme treatment).

The average fiber diameter of a fiber used for the hazardous substance removing material of the present invention is preferably 50 μm or less, more preferably 10 μm or less, particularly preferably 1 μm or less, and most preferably 100 nm or less. The average fiber diameter of the present invention is obtained by measuring the diameters of fibers in arbitrarily selected 300 sites on a scanning electron microscope (SEM) image for observation and averaging the results by calculation.

As to the method for producing the fiber used in the present invention, there are typical production methods such as melting spinning, wet spinning, dry spinning, and dry-wet spinning, and methods in which the fiber is made fine by a physical process (such as strong mechanical shearing using an ultrahigh pressure homogenizer), although dry spinning or dry-wet spinning is preferably employed for obtaining a stable quality of product. For producing an uniform fiber having an average fiber diameter of 100 nm or less, the electrospinning method disclosed in “Kakou Gijyutsu (Processing Technology)”, 2005, Vol. 40, No. 2, p. 101 and p. 167; “Polymer International”, 1995, Vol. 36, pp. 195-201; “Polymer Preprints”, 2000, Vol. 41(2), p. 1193; “Journal of Macromolecular Science: Physics”, 1997, B36, p. 169; and the like is preferably used.

Regarding the solvent used for the spinning, any solvent may be used as long as it dissolves the resin used for synthetic resin fibers. Examples thereof include: chloride-based solvents such as methylene chloride, chloroform, and dichloroethane; amide-based solvents such as dimethylformamide, dimethylacetamide, and N-methylpyrrolidone; ketone-based solvents such as acetone, ethyl methyl ketone, methyl isopropyl ketone, and cyclohexanone; ether-based solvents such as THF and diethyl ether, and alcohol-based solvents such as methanol, ethanol, and isopropanol. These solvents may be used either singularly, or in mixtures of a plurality of types thereof.

The resin solution used for the electrospinning method may be added with a salt such as lithium chloride, lithium bromide, potassium chloride, and sodium chloride.

Preferably, fibers constituting a carrier of the hazardous substance removing material of the present invention partially adhere to each other such that a structure forming a three-dimensional network is obtained. The use of such structure results in the improvement of mechanical tolerance upon processing or practical use, leading to the improvement of reliability of the hazardous substance removing material. Further, antibody-supporting properties of the present invention can be improved. Adhesion between fibers can be observed by a method involving SEM or the like. The density of fiber adhesion points is preferably 10 adhesion points or more in a 1-mm square on the projected surface area of the hazardous substance removing material and preferably 100 adhesion points or more in the same.

Regarding a method for forming adhesion points, adhesion points may be formed by a dry spinning method or by a melt spinning method. After spinning, adhesion point formation treatment may be carried out by heating or adding an adhesive/plasticizing solvent or the like. In view of production cost, it is preferable to form adhesion points by a dry spinning method with the use of an appropriate solution formulation.

### (2) Polymer Material Having Affinity for Fc Region of Antibody

In the hazardous substance removing material of the present invention, a polymer material having affinity for Fc region of antibody is supported on a carrier.

Antibody is a biological polymer that acts in the immunomechanism of a living body. There are 5 types of mammalian antibodies, namely, IgQ IgE, IgD, Ig, and IgA. An antibody corresponding to IgG contained in the yolk of Aves is referred to as IgY Any type of antibody may be used herein. As the antibody used in the present invention, IgG and IgY are preferable, in that they are supported on a substrate, and in that the concentrations thereof in serum or yolk are high and they are easily used. IgY is particularly preferable in that the total production amount per animal is high and purification is easy.

The term “polymer material having an affinity for Fc region of the antibody” is used in the present invention to mean a polymer material capable of forming a complex with the Fc region of an antibody in the state of a homogenous aqueous solution. Such formation of a complex in the state of a homogenous aqueous solution can be confirmed, for example, by a method comprising fluorescently labeling both a polymer material and a fragment in the Fc region of an antibody separated by the treatment of the antibody with papain or pepsin and chromatography, mixing them, and separating the mixture by chromatography, followed by observation under a fluorescence microscope; or a method comprising supplying an antibody Fc fragment or a polymer material to a micro flow channel on which the other component is immobilized, and then confirming such formation of a complex by fluorescence measurement, an SPR method, a QCM method, calorimetry, a spectroscopic method, etc.

The polymer material having an affinity for the Fc region of an antibody used in the present invention is preferably at least one type selected from the group consisting of protein A, protein G, and lectin.

Protein A is a protein with a molecular weight of approximately 42,000, which accounts for 5% of the cell wall ingredient of a Gram-positive bacterium, Staphylococcus aureus. This protein has a characteristic feature of specifically binding to the Fc fragment of immunoglobulin G (IgG) of a human, a mouse, a rabbit, and the like. The protein A of the present invention may be either a native protein or a recombinant protein. Protein A generally has resistance to high temperatures. Thus, even after protein Ahas been exposed to 4 M urea or 6 M guanidine hydrochloride, it is able to maintain a native conformation. Its affinity for IgG largely differs depending on the type of the IgG or subclass, and further it is pH-dependent. For example, with regard to human-derived IgQ, protein A strongly binds to IgG1, IgG2 and IgG4, but it does not bind to IgG3. With regard to mouse-derived IgG, it strongly binds to IgG2a, IgG2b and IgG3 under standard buffer conditions such as Tris-HCl or PBS, but it only weakly binds to IgG1. Protein A hardly binds to rat IgG, regardless of subclass.

Protein G is a protein derived from the cell wall of Streptococcus (group G), and it binds to the IgG of almost all mammals. Native type protein G mainly binds to the Fc portion of an antibody, but it weakly binds also to Fab. In addition, from the DNA sequence thereof, it is found that it comprises not only two IgG-binding sites, but also sites binding to albumin or cell surface. An example of recombinant protein G is one which is produced by eliminating albumin-binding sites or sites that bind to cell surfaces to enhance specificity for IgG, so that it can be used in elimination of albumin from serum containing human IgG Protein G has been widely used in purification of monoclonal or polyclonal IgG, which does not bind (or only weakly binds to) protein A. The protein G of the present invention may be either a native type protein or a recombinant protein.

It is generally said that protein A and protein F have a low affinity for yolk-derived IgG (IgY). Although this point is problematic when such proteins are applied to an affinity column for purification, it is effective for the present invention. However, in order to efficiently immobilize such protein on the antibody Fc portion, it is desired to previously degrease IgY to be supported. It is preferably 10% or less, more preferably 5% or less, and most preferably 1% or less, with respect to IgY.

The term “lectin” means proteins other than enzymes or antibodies, which have ability to specifically recognize a sugar chain and to bind to a sugar chain portion of a glycoprotein or glycolipid. The lectin is contained in a large amount in plant seeds or the tissue fluids of animals, and it has a characteristic feature of acting on the surface of a cell membrane to activate a cell and suppressing the growth of bacteria. It also has functions of helping immune system or coagulating erythrocytes. Lectin is contained in a large amount in foods such as soybeans, potatoes, bush beans, and the like. Even in a case in which lectin has only one sugar-recognizing site in an intramolecular subdomain, it forms multimers in many molecules. Thus, many lectins have cross-link formation ability via sugar chain molecules.

Examples of animal lectin include: type-C lectin including selectin, collectin, etc.; type-S lectin comprising galectin; type-P lectin having a mannose-6-phosphate receptor, type-I lectin including a siglec family; calnexin; calreticulin; and calmegin.

Among plant lectins, lectins found in the seeds of leguminous plants have been vigorously studied. Examples of such lectins found in the seeds of leguminous plants include Conavalia ensiformis (ConA), Lens Culinariis (LCA), Bowringia midbraedii (BMA), Dolichos lablab (DLA), Galanthus nivalis (GNA), Geradia savaglia (GSL), Machaerium biovulatum (MBA), Machaeriumu lunatus (MLA), Narcissus pseudonarcissus (NPA), Epipactis heleborine (EHA), and Listera ovata (LOA). Examples of lectins derived from plants other than leguminous plants include wheat germ lectin (WGA), peanut lectin (PNA), potato lectin (STA), Datura tatula lectin (DAA), modeccin, abrin, and ricin regarding which Viscumalbum-derived mistletoe lectin and the like are classified into a similar group.

Like the aforementioned calnexin and calreticulin, there are also lectins, which are not only derived from multicellular eukaryotes, but also derived from several types of yeasts.

Bacterial toxins such as cholera toxin, enterotoxin of Escherichia coli, pertussis toxin, Shiga toxin, and verotoxin derived from Escherichia coli are also classified into lectins.

The type of lectin used in the present invention is not particularly limited, as long as it has an affinity for the Fc portion of an antibody. Among others, mannose-binding-type lectin is preferable. The term “mannose-binding-type lectin” is used in the present invention to mainly mean lectin that recognizes an α-mannosyl residue as a constituent sugar of mother nucleus of an asparagine-binding sugar chain.

Examples of mannose-binding-type lectins derived from animals include ERGIC-53, VIP36, and MBP. Examples of mannose-binding-type lectins derived from plants include snowdrop (GNA) bulb lectin, Amarylidacaceae-derived lectin, Liliaceae-derived lectin, Orchidales-derived lectin, pea lectin, concanavalin A (ConA), soybean lectin, lentil lectin, and seaweed-derived lectin (ESA).

From the viewpoint of function, availability, performance stability, and economic efficiency, lectins contained in the seeds of leguminous plants are preferably used in the present invention. Among others, ConA is particularly preferably used.

The polymer material of the present invention having an affinity for the Fc region of an antibody is added at a mass percentage of preferably 0.1% to 1,000%, more preferably 1% to 500%, and most preferably 5% to 200%, with respect to the mass of the antibody.

It is preferable that the surface of a carrier be coated with the polymer material having an affinity for the Fc region of an antibody. The average thickness of a layer comprising the polymer material having an affinity for the Fc region of an antibody is preferably between 5 and 20 nm.

The polymer material having an affinity for the Fc region of an antibody not only enables effective utilization of an antigen-recognizing site, but it can also exhibit functions such as provision of a hydrophilic site.

### (3) Hydrophilic Polymer

In the present invention, a hydrophilic polymer as well as a polymer material having affinity for Fc region of antibody can be supported on a carrier. The hydrophilic polymer that can be used in the present invention means a polymer having a hydrophilic functional group in the structure thereof. The type of the hydrophilic functional group is not particularly limited. A polymer containing at least one type selected from among a hydroxyl group, an amino group, an amide group, a carboxylic acid group, and a quaternary amino group is preferable. A polymer having an amino group, an amide group, and a quaternary amino group is most preferable. Examples of a polymer having a hydroxyl group include polyvinyl alcohol, a polyethylene-polyvinyl alcohol copolymer, a partial hydrolysate of vinyl polyacetate, and partially substituted cellulose derivatives such as diacetyl cellulose, ethyl cellulose or carboxymethyl cellulose. In addition, natural products such as guar gum, pectin, starch, carrageenan, glucomannan or sialyllactose, or the synthetic products thereof may also be included. Of these examples, polyvinyl alcohol is preferable. Examples of a polymer having an amino group include polyvinylamine, and polyaminocaproic acid methacrylate. In addition, natural products such as chitosan or the synthetic products may also be included. Of these examples, polyvinylamine is preferable.

Examples of a polymer having an amide group include single polymers such as polyacrylamide or polyvinylpyrrolidone and copolymers consisting of such polymer and (meth)acrylate or a vinyl polymer such as vinyl acetate. In addition, natural products such as collagen, gelatin, fibroin, casein or kelatin, or the synthetic products thereof may also be included. (The amide group of the present invention may also include an amide group that constitutes a peptide bond.) Of these examples, polyacrylamide, polyvinylpyrrolidone, and gelatin are preferable.

Examples of a polymer having a polycarboxylic acid group include polyacrylic acid, carboxymethyl cellulose, and polylactic acid. In addition, natural products such as alginic acid or hyaluronic acid, and the synthetic products thereof may also be included. Of these examples, polyacrylic acid is preferable. A part of or the entire carboxylic acid group may be in an undissociated state, or it may form the salts of sodium, potassium, ammonium, and the like.

A cationic polymer is also preferably used. A quaternary ammonium salt group is obtained by adding halogenated alkyl or the like to an alkylamino group. Specific examples of a monomer that derives a constituent unit having a quaternary ammonium group include an N,N-dimethylaminoethyl(meth)acrylate methyl chloride quaternary product, an N,N-diethylaminopropyl(meth)acrylamide methyl chloride quaternary product, and an N,N-dallylmethylamine methyl chloride quaternary product Other examples of a cationic polymer include polydiallyldimethylammonium chloride, polyethyleneimine, a polyvinylpyridine quaternary salt, and a polymer having a quaternary phosphonium group. Moreover, other examples of such cationic polymer also include copolymers of these compounds and condensation products such as dicyandiamide with formalin or alkylenediamine with epichlorohydrin. Furthermore, a betaine polymer having such cationic group and an anionic group such as carboxylic acid, sulfonic acid or phosphonic acid may also be used.

The molecular weight of a hydrophilic polymer that can be used in the present invention may be arbitrarily determined depending on the type thereof, the purpose thereof, the kind of a supported antibody, and the like. In general, the weight-average molecular weight of such hydrophilic polymer is preferably 5,000 to 1,000,000, more preferably 10,000 to 500,000, and most preferably 30,000 to 300,000. The content of a hydrophilic group in the hydrophilic polymer of the present invention may be arbitrarily determined depending on the type thereof, the purpose thereof, the kind of a supported antibody, and the like. The content is preferably between 0.1 to 3 groups, more preferably between 0.3 to 1.5 groups, and most preferably 0.5 to 1 group, per monomer unit. A polymer used in coating may be used singly. Otherwise, several polymers may be mixed, or it may be used as a copolymer with any given monomer. The fact that the hydrophilic polymer of the present invention should be selected from the viewpoint of affinity, not only for an antibody, but also for a substrate material, is obvious to persons skilled in the art. That is to say, a preferred hydrophilic polymer differs depending on the type of a product selected as a substrate. From this viewpoint, a compound having a high affinity for a substrate may be mixed with the aforementioned hydrophilic polymer at any given ratio, and the mixture may be then used. Otherwise, a compound having a high affinity for a substrate may be copolymerized with the aforementioned hydrophilic polymer, and the mixture may be then used. The mixing ratio between the polymer material having affinity for Fc region of antibody and a hydrophilic polymer in the present invention is 1:1 to 1:100, preferably 1:1 to 1:20, and most preferably 1:2 to 1:10.

### (4) Antibody

The antibody used for the hazardous substance removing material of the present invention is a protein, which is reactive (antigen-antibody reaction) specifically to a specific hazardous substance (antigen), has a molecule size of 7 to 8 nm, and is in a Y-shaped molecular form. In the Y-shape molecular structure of the antibody, a pair of branch portions of the antibody are called Fab, and a stem portion thereof is called Fc. Among them, the Fab portions capture the hazardous substance.

The type of the aforementioned antibody corresponds to the type of the hazardous substance to be captured. Examples of the hazardous substance to be captured by the antibody include bacteria, fungi, viruses, allergens, and mycoplasmas. Specifically, the bacteria include, for example: the genus Staphylococcus (such as Staphylococcus aureus and Staphylococcus epidermidis), Micrococcus, Bacillus anthracis, Bacillus cereus, Bacillus subtilis, and Propionibacterium acnes, as gram-positive bacteria; and Pseudomonas aeruginosa, Serratia marcescens, Burkholderia cepacia, Streptococcus pneumoniae, Legionella pneumophilia, and Mycobacterium tuberculosis, as gram-negative bacteria. The fungi include, for example, Aspergillus, Penicillius, and Cladosporium. The viruses include influenza viruses, coronavirus (SARS virus), adenovirus, and rhinovirus. The allergens include pollens, mite allergens, and cat allergens.

In particular, in the present invention, an influenza antibody, which involves droplet infection and becomes a target of a hazardous substance removing filter, can be preferably used. As an antigen used in production of such influenza antibody, antigens such as type H1N1 virus antigen, type H3N2 virus antigen, and type B virus antigen, a triple antigen, and a H5 recombinant protein derived from avian influenza virus H5N1 can be used. The H5 recombinant protein kills chickens. Thus, in the case of this protein, an antibody cannot be obtained from a chicken egg. However, it is possible to immunize an ostrich with this recombinant protein.

Examples of a method for producing the aforementioned antibody include: a method in which an antigen is administered to an animal such as a goat, a horse, a sheep, and a rabbit, and a polyclonal antibody is purified from the blood thereof; a method in which splenic cells of an animal to which an antigen has been administered and cultured cancer cells are subjected to cell fusion and a monoclonal antibody is purified from a culture solution thereof or from a body fluid (such as ascites) of an animal in which the fussed cells have been implanted; a method in which an antibody is purified from a culture solution of genetically modified bacteria, plant cells, or animal cells into which an antibody-producing gene has been introduced; and a method in which an ostrich or a chicken to which an antigen has been administered is allowed to lay an immune egg, and an ostrich egg antibody or a chicken egg antibody is purified from yolk powders obtained by sterilizing and spray-dying the yolk of the immune egg. Of all the above methods, the method for obtaining the antibody from an ostrich egg or a chicken egg enables easy mass production of the antibody, reducing the cost of the hazardous substance removing material.

The antibody used for the hazardous substance removing material of the present invention is preferably an antibody produced from an ostrich or chicken egg.

As an antibody produced from an ostrich egg, that described in International Publication WO2007/026689 can be used, for example. According to a method using an ostrich egg, an antibody specific for a protein, which has been hardly produced by the conventional methods, can be easily produced. Thus, a large amount of homogenous antibody can be produced with no difference in lots. The term “ostrich” is used to mean Aves belonging to Struthioniformes. Among other, Struthio camelus belonging to Struthionidae is preferably used. An antibody can be produced from an ostrich egg according to the method described in paragraphs [0007] to [0034] of International Publication WO2007/026689.

It is desirable that the carrier constituting the hazardous substance removing material of the present invention is subjected to antibacterial treatment such as coating of an agent containing an antibacterial agent and/or antifungal treatment such as coating of an agent containing an antifungal agent The antibody is principally a protein, and particularly, the ostrich egg antibody is food, and the antibody may also accompany a protein other than the antibody. These proteins might serve as food for bacteria and fungi to proliferate. However, if the carrier is subjected to antibacterial and/or antifungal treatment, such multiplication of bacteria and the fungi is suppressed, so that a long-term storage becomes possible.

The antibacterial/antifungal agents include organic silicon quaternary ammonium salts, organic quaternary ammonium salts, biguanides, polyphenols, chitosan, silver-support colloidal silica, zeolite-support silvers, and the like. As to the treatment method, there are a post-treatment method in which an antibacterial/antifungal agent is immersed in or applied to the support made of a fiber, a raw thread/raw cotton improving method in which an antibacterial/antifungal agent is mixed in the step of synthesizing a fiber constituting the carrier, and the like.

Regarding methods for immobilizing the antibody to the carrier, there are: a method in which, after a carrier is subjected to silane treatment using γ-aminopropyl-triethoxysilane or the like, an aldehyde group is introduced on the surface of the carrier by glutraldehyde or the like, to effect a covalent bond between the aldehyde group and an antibody; a method in which an untreated carrier is immersed into an aqueous solution of an antibody to cause ion boding, thereby immobilizing the antibody to the carrier, a method in which an aldehyde group is introduced to a carrier having a specific functional group to effect a covalent bond between the aldehyde group and an antibody; a method in which a carrier having a specific functional group is ion-bonded to an antibody; and a method in which a carrier is coated with a polymer having a specific functional group, followed by an introduction of an aldehyde group to effect a covalent bond between the aldehyde group and an antibody. In the present invention, antibody can be simply supported on a carrier by spraying a solution of antibody onto a carrier.

The hazardous substance removing material of the present invention can be used for a filter for an air purifier, a mask, a wipe sheet, and the like.

When the hazardous substance removing material of the present invention is used for an air purifier filter, it may be used in combination with the following conventional filters and any other conventional filters: a prefilter for removing dusts, a dust removal filter, a photocatalyst filter having deodorant effects, an antibacterial filter for removing other hazardous substances, and a VOC-absorbing filter.

### EXAMPLES

The features of the present invention are hereafter more specifically described with reference to examples and comparative examples. Materials, their quantities consumed, proportions thereof, contents of processing, processing procedures, and the like set forth in the following examples can be appropriately modified without departing from the sprit of the present invention. Accordingly, the scope of the present invention is not to be construed as being limited to the specific examples shown below.

### Example 1

### Production of Carrier

An acetone/water (97:3) solution containing cellulose acetate (total degree of substitution: 2.4; number average molecular weight: 30,000; manufactured by Aldrich) (25% by mass) was heated to 60° C. and then was squirted with air out of a nozzle of 0.1 mm in diameter at a spinning rate of 500 m/m for nonwoven fabric formation. Accordingly, a nonwoven fabric N−1 with a membrane thickness of 85 μm was obtained. A spinning cylinder was heated to 100° C. with a heater. The average fiber diameter was measured by SEM, and it was found to be 8 μm.

### (Production of Antibody)

0.5 mL of an antigen solution containing inactivated influenza virus (100 μg) was mixed with 0.5 mL of a complete adjuvant. The obtained mixture was inoculated into the chest muscle of an ostrich for an initial immunization. For a second and the subsequent immunizations, 0.5 mL of the same above antigen solution was mixed with 0.5 mL of an incomplete adjuvant, and the obtained mixture was inoculated into the neck muscle of the ostrich every one week until the fourth week. Only yolk was collected from an egg laid by this ostrich, and it was then stirred. 10 mL of this yolk solution was mixed with TBS (20 mM Tris-HCL (pH 7.5), 0.15 M NaCL, 0.5% NaN), and 5 mL of 10% dextran sulfate/TBS was then added to the mixture. The thus obtained mixture was stirred for 30 minutes. 10 mL of 1 M CaCl₂/TBS was added to the reaction solution, and the obtained mixture was then stirred. The reaction solution was then left at rest for 2 hours or more. Thereafter, the resultant was centrifuged at 10,000 rpm for 30 minutes, and a supernatant was then recovered. Ammonium sulfate was added to the supernatant to a final concentration of 40%, and the mixture was then left at rest for 12 hours or more. The resultant was centrifuged at 10,000 rpm, and a precipitate was then recovered. This precipitate was resuspended in 10 mL of TBS, and it was then dialyzed against TBS.

### (Production of Filter Sample)

Pea lectin (manufactured by Wako Pure Chemical Industries, Ltd.) was diluted to a concentration of 100 ppm, and it was then uniformly developed on a 10-cm² nonwoven fabric sample N−1, followed by drying with air-blowing at 40° C. for 2 hours, so as to produce a substrate sample.

Subsequently, 1 mL of a coating solution prepared by diluting the aforementioned dialyzed solution with water to result in an antibody concentration of 100 ppm was uniformly developed on the aforementioned 10-cm² substrate sample, and it was then left at rest at room temperature for 1 hour. Thereafter, the resultant was dried with air-blowing at 40° C. for 2 hours, so as to produce a filter sample (this coating method is referred to as a “two-step coating method”).

On the other hand, 1 mL of a mixed solution of pea lectin and an antibody (which had been prepared so that each concentration had become 100 ppm) was developed on a 10-cm² nonwoven fabric sample, and it was then air-dried at 40° C. for 2 hours, so as to produce a filter sample (this coating method is referred to as a “one-step coating method”).

Moreover, filter samples were produced by the same above method with the exception that pea lectin was replaced with the samples shown in Table 1. Furthermore, standard products, in which only gelatin was used, were also evaluated as comparative examples (sample Nos. 10 and 11).

Samples Nos. 8 and 9 were prepared so that the concentrations of pea lectin and gelatin became each 100 ppm.

Protein A: manufactured by Pierce Biotechnology

Protein G: manufactured by Pierce Biotechnology

Gelatin: manufactured by MP Biomedicals; derived from bovine skin

Filter samples were prepared by the same above method with the exception that pea lectin was replaced with the samples shown in Table 2. As a coating method, only the aforementioned two-step coating method was applied.

Concanavalin A (ConA): manufactured by Wako Pure Chemical Industries, Ltd.

Soybean lectin: manufactured by Wako Pure Chemical Industries, Ltd.

Lentil lectin: manufactured by Wako Pure Chemical Industries, Ltd.

Wheat germ agglutinin (lectin): manufactured by Wako Pure Chemical Industries, Ltd.

Peanut lectin: manufactured by Wako Pure Chemical Industries, Ltd.

### (Elisa Measurement)

Each of the aforementioned filters was cut into a weight of 0.1 to 1.0 mg (10 samples for each standard), and it was then disposed on a 96-well immuno plate manufactured by Nunc. Subsequently, BlockAce (manufactured by Dainippon Pharma Co., Ltd.) was mixed with PBS(−1) at a ratio of 1:1 to prepare a blocking solution, and 200 μL of the blocking solution was added to the aforementioned filter, and it was then left at rest at 37° C. for 1 hour, so as to carry out a blocking treatment. As a washing solution, PBS(−) containing 0.05% TWEEN20 was used. Hereafter, washing operations were carried out 3 times each between individual steps. Subsequently, an influenza vaccine antigen (manufactured by the Kitazato Institute) was poured thereto, and it was then left at rest at 37° C. for 1 hour. Thereafter, a 20,000-fold diluted solution (PBS(−)) of an HRP-labeled antibody of anti-influenza virus IgG (manufactured by AbD) was poured thereto, and it was then left at rest at 37° C. for 1 hour. Thereafter, 3,3′,5,5′-tetramethylbenzidine (TMB; manufactured by Sigma) was poured thereto, and it was then left at rest for 15 minutes in a dark place. Thereafter, a termination solution (0.5 mol/L sulfuric acid) was poured thereto, and it was then stirred for 1 minute. 100 μL of the reaction solution was extracted, and it was then placed into another immuno plate. Thereafter, the absorbance at 450 nm (control: 620 nm) was measured with a Microplate Reader (manufactured by Bio-Rad Laboratories). It was confirmed that the degree of color development of an antibody-non-supported sample was significantly low, and that a blocking treatment was properly carried out. A comparison was made among the samples in terms of color density per unit weight.

(Note: Since the weight of an antibody and that of a polymer material were small enough to that of a substrate (up to ppm order), they are negligible.)

### (Evaluation of Preservative Quality)

The aforementioned filter was left at rest for 1 week in an environment of 50° C. and 90% RH. The thus obtained sample was also subjected to the same evaluation.

### (Airborne Bacteria-Capturing Experiment)

Staphylococcus aureus (ATCC) was inoculated into 100 mL of soybean casein, and it was then incubated at 37° C. for 24 hours, while gently sting. Thereafter, the reaction solution was diluted with peptone water, so as to obtain a sample solution of approximately 1E6 CFU. Aerosol produced by spraying the sample solution with a nebulizer was applied to a filter sample of 8 cm×8 cm at a flow rate of 1.4 L/min for 1 minute, and the titer of a solution recovered with a glass impinger was then examined by a standard plaque assay method, so as to evaluate a bacteria-capturing rate (wherein the capturing rate obtained from the results of a control test using no filters was defined as 0%).

TABLE 1

| | | | Color density per mg of filter sample | | Staphylococcus | |
|---|---|---|---|---|---|---|
| Sample | Polymer | Coating | Immediately | After preservation at | aureus- capturing | |
| No. | material | method | after production | 50° C., 90% RH, 7 days | rate (%) | Remarks |
| 1 | None | — | 0.68 | 0.39 | 6.0 | Comparative |
| | | | | | | example |
| 2 | Pea lectin | Two-step | 1.62 | 1.55 | 32.5 | The present |
| | | coating | | | | invention |
| 3 | Pea lectin | One-step | 1.38 | 1.35 | 30.3 | The present |
| | | coating | | | | invention |
| 4 | Protein A | Two-step | 1.34 | 1.26 | 25.8 | The present |
| | | coating | | | | invention |
| 5 | Protein A | One-step | 1.08 | 1.05 | 23.2 | The present |
| | | coating | | | | invention |
| 6 | Protein G | Two-step | 1.28 | 1.20 | 23.8 | The present |
| | | coating | | | | invention |
| 7 | Protein G | One-step | 0.98 | 0.96 | 20.9 | The present |
| | | coating | | | | invention |
| 8 | Pea lectin + | Two-step | 1.55 | 1.49 | 35.4 | The present |
| | gelatin | coating | | | | invention |
| 9 | Pea lectin + | One-step | 1.26 | 1.25 | 31.0 | The present |
| | gelatin | coating | | | | invention |
| 10 | Gelatin | Two-step | 0.69 | 0.56 | 9.1 | Comparative |
| | | coating | | | | example |
| 11 | Gelatin | One-step | 0.59 | 0.48 | 8.7 | Comparative |
| | | coating | | | | example |

TABLE 2

| | | Color density per mg of filter sample | | |
|---|---|---|---|---|
| Sample | | Immediately | After preservation at 50° C., | |
| No. | Polymer material | after production | 90% RH, 7 days | Remarks |
| 101 | None | 0.69 | 0.38 | Comparative example |
| 102 | Pea lectin | 1.63 | 1.57 | The present invention |
| 103 | ConA | 1.81 | 1.75 | The present invention |
| 104 | Soybean lectin | 1.66 | 1.60 | The present invention |
| 105 | Lentil lectin | 1.71 | 1.65 | The present invention |
| 106 | Wheat germ lectin | 1.35 | 1.24 | The present invention |
| 107 | Peanut lectin | 1.55 | 1.47 | The present invention |
| 108 | ConA + gelatin | 1.70 | 1.64 | The present invention |
| 109 | Gelatin | 0.57 | 0.46 | Comparative example |

From the results shown in Table 1, it was found that the embodiment of the present invention brings on a large amount of antigen captured per antibody, and thus that a supported antibody can be efficiently used. In addition, it was confirmed that the present invention enables the improvement of the preservative quality of a supported antibody and the improvement of an airborne bacteria-capturing rate.

As is clear from the results shown in Table 2, in comparison with samples Nos. 106 and 107, the amount of an antigen captured per antibody is high in samples 102, 103, 104, and 105. Thus, it was found that it is particularly advantageous to use mannose-binding-type lectin contained in the seeds of leguminous plants as the polymer material used in the present invention.

1. A hazardous substance removing material consisting of a carrier on which an antibody and a polymer material having an affinity for Fc region of the antibody are supported.

2. The hazardous substance removing material according to claim 1, wherein the polymer material is at least one type selected from the group consisting of protein A, protein G, and lectin.

3. The hazardous substance removing material according to claim 1, wherein the polymer material is lectin.

4. The hazardous substance removing material according to claim 1, wherein the polymer material is mannose-binding-type lectin.

5. The hazardous substance removing material according to claim 1, wherein the surface of the carrier is coated with the polymer material.

6. The hazardous substance removing material according to claim 1, which has a hydrophilic polymer as well as the polymer material on the carrier.

7. The hazardous substance removing material according to claim 6, wherein the hydrophilic polymer has a hydroxyl group, an amino group, an amide group, a carboxylic acid group, or a quaternary amino group.

8. The hazardous substance removing material according to claim 1, which is obtained by forming a layer comprising a polymer material having an affinity for Fc region of an antibody on a carrier and then supporting the antibody on the carrier.

9. The hazardous substance removing material according to claim 1, wherein the antibody is derived from ostriches.

10. A method for removing hazardous substance, which comprises removing a hazardous substance from a gas phase or a liquid phase using a hazardous substance removing material consisting of a carrier on which an antibody and a polymer material having an affinity for Fc region of the antibody are supported.
